(19) United States
(12) Patent Application Publication
(10) Pub. No.: US 2018/0155255 A1
(43) Pub. Date: Jun. 7, 2018

(54) Process of Producing Paraxylene by The Methylation of Toluene and/or Benzene

(71) Applicant: ExxonMobil Chemical Patents Inc., Baytown, TX (US)

(72) Inventors: Todd E. Detjen, Houston, TX (US); Robert G. Tinger, Friendswood, TX (US)

(73) Assignee: ExxonMobil Chemical Patents Inc., Baytown, TX (US)

(21) Appl. No.: 15/797,515

(22) Filed: Oct. 30, 2017

Related U.S. Application Data
(60) Provisional application No. 62/430,021, filed on Dec. 5, 2016.

(30) Foreign Application Priority Data
Jan. 9, 2017 (EP) 17150624.9

(51) Int. Cl. C07C 2/86

(52) U.S. Cl. CPC C07C 2/864

(57) ABSTRACT

A process for producing paraxylene by the catalytic alkylation of benzene and/or toluene with methanol. In prior art processes, water is typically co-injected with the methanol to improve the utilization of methanol, increase the amount of methanol that reacts with the benzene and/or toluene, and decrease the amount of methanol that decomposes to undesirable carbon monoxide, carbon dioxide, or water or reacts with itself to produce unwanted light olefinic gases. Rather than using purified methanol and co-feeding water as is taught in the prior art, crude, or unpurified, methanol that contains at least 5 wt %, such as between 5 and 35 wt %, water, based on the total amount of water and methanol feed, can be used as the alkylating agent, reducing the need to co-inject water at least partially, if not completely.

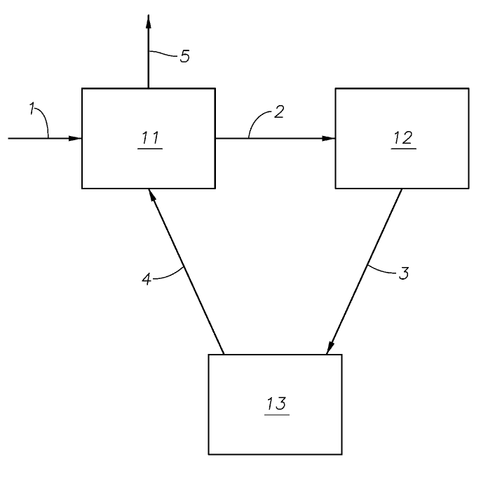

### PRIORITY CLAIM

This application claims priority to and the benefit of U.S. Provisional Patent Application Ser. No. 62/430,021, filed Dec. 5, 2016 and EP 17150624.9, filed Jan. 9, 2017, the contents of each being incorporated herein by reference in their entireties.

### TECHNICAL FIELD

This disclosure relates to a process for using unpurified methanol in processes to produce gasoline and aromatics. More particularly, unpurified methanol is used as a methylating agent in the production of paraxylene by the alkylation of benzene and/or toluene with methanol.

### BACKGROUND

Of the xylene isomers, paraxylene is of particular value since it is useful in the manufacture of terephthalic acid, which is an intermediate in the manufacture of synthetic fibers and resins. Today, paraxylene is commercially produced by hydro-treating of naphtha (catalytic reforming), steam cracking of naphtha or gas oil, and toluene disproportionation.

One problem with most existing processes for producing xylenes is that they produce a thermodynamic equilibrium mixture of ortho (o)-, meta (m)- and para (p)-xylenes, in which the paraxylene concentration is typically only about 24 wt %. Thus, separation of paraxylene from such mixtures tends to require superfractionation and multistage refrigeration steps. Such processes involve high operating and capital costs and result in only limited yields. Therefore, there is a continuing need to provide processes which are highly selective for the production of paraxylene.

It is well-known to manufacture xylenes by the alkylation of toluene and/or benzene with methanol, and, in particular, to selectively make paraxylene (PX) product using zeolite catalyst. See, for instance, U.S. Pat. Nos. 4,002,698; 4,356,338; 4,423,266; 5,675,047; 5,804,690; 5,939,597; 6,028,238; 6,046,372; 6,048,816; 6,156,949; 6,423,879; 6,504,072; 6,506,954; 6,538,167; and 6,642,426. The terms “paraxylene selectivity”, “para-selective”, and the like, means that paraxylene is produced in amounts greater than is present in a mixture of xylene isomers at thermodynamic equilibrium, which at ordinary processing temperatures is about 24 mol %. Paraxylene selectivity is highly sought after because of the economic importance of paraxylene relative to meta- and orthoxylene. Although each of the xylene isomers have important and well-known end uses, paraxylene is currently the most economically valuable.

In the process, typically toluene and/or benzene are alkylated with methanol, in the presence of a suitable catalyst, to form xylenes in a reactor in a system illustrated schematically in the FIGURE, wherein a feed comprising reactants enter fluid bed reactor 11 via conduit 1 and effluent comprising product exits through conduit 5, and the catalyst circulates between fluid bed reactor 11, apparatus 12, which strips fluid from the catalyst, and catalyst regenerator 13, via conduits 2, 3, and 4, respectively. Water is typically co-fed with toluene and methanol to minimize toluene coking in the feed lines and methanol self-decomposition. Other side reactions include the formation of light olefins, light paraffins, as reactions that convert paraxylene to other xylene isomers or heavier aromatics.

It is desirable to continue to improve the process and save energy and costs.

### BRIEF SUMMARY

Embodiments disclosed herein provide a process for producing paraxylene by the catalytic alkylation of benzene and/or toluene with methanol. In prior art processes, water is typically co-injected with the methanol to improve the utilization of methanol, increase the amount of methanol that reacts with the benzene and/or toluene, and decrease the amount of methanol that decomposes to undesirable carbon monoxide, carbon dioxide, or water, or reacts with itself to produce unwanted light olefinic gases. Rather than using purified methanol and co-feeding water as is taught in the prior art, crude or unpurified methanol that contains at least 5 wt %, such as between 5 and 35 wt %, water, based on the total amount of water and methanol feed, can be used as the alkylating agent, reducing the need to co-inject water at least partially, if not completely. Thus, it should be appreciated that in at least some embodiments no additional water is co-injected along with the unpurified methanol. Using unpurified methanol that contains at least 5 wt % water is beneficial because it reduces the costs associated with purchasing the raw materials and saves capital and energy on the methanol production step.

In one embodiment, toluene and/or benzene is contacted with an alkylating agent, in the presence of an alkylation catalyst in an alkylation reactor under alkylation conditions to produce an alkylation effluent comprising paraxylene. Unpurified methanol that contains at least 5 wt % of water, based on the weight of the unpurified methanol, is used as the alkylating agent. Paraxylene may then be recovered from the alkylation effluent.

The use of unpurified methanol may also be useful in other processes to produce gasoline and aromatics, such as methanol-to-gasoline processes or methanol-to-aromatics processes.

### BRIEF DESCRIPTION OF THE DRAWING

The FIGURE is a schematic of a reactor system including reactor and regenerator and some associated auxiliary devices and transfer piping per se known in the art.

### DETAILED DESCRIPTION OF THE EMBODIMENTS

Described herein is a process for producing paraxylene by the catalytic alkylation of benzene and/or toluene with methanol. In prior art processes, water is typically co-injected with the methanol to improve the utilization of methanol, increase the amount of methanol that reacts with the benzene and/or toluene, and decrease the amount of methanol that decomposes to undesirable carbon monoxide, carbon dioxide, or water, or reacts with itself to produce unwanted light olefinic gases. Rather than using purified methanol and co-feeding water as is taught in the prior art, crude or unpurified methanol that contains at least 5 wt %, such as between 5 and 35 wt %, water, based on the total amount of water and methanol feed, can be used as the alkylating agent, reducing the need to co-inject water at least partially, if not completely. Thus, it should be appreciated that in at least some embodiments no additional water is co-injected along with the unpurified methanol. Using unpurified methanol that contains at least 5 wt % water is beneficial because it reduces the costs associated with purchasing the raw materials and saves capital and energy on the methanol production step.

As used herein, “crude methanol” or “unpurified methanol” means methanol that has not been processed through at least one of the purifying fractionation towers in a methanol production plant. The actual composition of the unpurified methanol will vary based upon the production process and what, if any, purification steps have been performed. “Crude methanol” and “unpurified methanol” are used interchangeably herein.

The alkylation process employed herein can employ any aromatic feedstock comprising benzene and/or toluene, although in general it is preferred that the aromatic feed contains at least 90 wt %, especially at least 99 wt %, of toluene. The process may be conducted in one or more fixed, moving, or fluidized bed rectors and employ any catalyst system known in the art.

In a particular embodiment, the catalyst employed in the alkylation process is generally a porous crystalline material and, in one preferred embodiment, is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa).

As used herein, the Diffusion Parameter of a particular porous crystalline material is defined as D/r²×10⁶, wherein D is the diffusion coefficient (cm²/sec) and r is the crystal radius (cm). The diffusion parameter can be derived from sorption measurements provided the assumption is made that the plane sheet model describes the diffusion process. Thus, for a given sorbate loading Q, the value Q/Q_{eq}, where Q_{eq} is the equilibrium sorbate loading, is mathematically related to (Dt/r²)^{1/2} where t is the time (sec) required to reach the sorbate loading Q. Graphical solutions for the plane sheet model are given by J. Crank in “The Mathematics of Diffusion”, Oxford University Press, Ely House, London, 1967.

The porous crystalline material is preferably a medium-pore size aluminosilicate zeolite. Medium pore zeolites are generally defined as those having a pore size of about 5 to about 7 Angstroms, such that the zeolite freely sorbs molecules such as n-hexane, 3-methylpentane, benzene, and paraxylene. Another common definition for medium pore zeolites involves the Constraint Index test which is described in U.S. Pat. No. 4,016,218, which is incorporated herein by reference. In this case, medium pore zeolites have a Constraint Index of about 1-12, as measured on the zeolite alone without the introduction of oxide modifiers and prior to any steaming to adjust the diffusivity of the catalyst. In addition to the medium-pore size aluminosilicate zeolites, other medium pore acidic metallosilicates, such as silicoaluminophosphates (SAPOs), can be used in the present process.

Particular examples of suitable medium pore zeolites include ZSM-5, ZSM-11, ZSM-12, ZSM-22, ZSM-23, ZSM-35, and ZSM-48, with ZSM-5 and ZSM-11 being particularly preferred. In one embodiment, the zeolite employed is ZSM-5 having a silica to alumina molar ratio of at least 250, as measured prior to any treatment of the zeolite to adjust its diffusivity.

Zeolite ZSM-5 and the conventional preparation thereof are described in U.S. Pat. No. 3,702,886. Zeolite ZSM-11 and the conventional preparation thereof are described in U.S. Pat. No. 3,709,979. Zeolite ZSM-12 and the conventional preparation thereof are described in U.S. Pat. No. 3,832,449. Zeolite ZSM-23 and the conventional preparation thereof are described in U.S. Pat. No. 4,076,842. Zeolite ZSM-35 and the conventional preparation thereof are described in U.S. Pat. No. 4,016,245. ZSM-48 and the conventional preparation thereof are taught by U.S. Pat. No. 4,375,573. The entire disclosures of these U.S. patents are incorporated herein by reference.

The medium pore zeolites described above are preferred for the present process since the size and shape of their pores favor the production of paraxylene over the other xylene isomers. However, conventional forms of these zeolites have Diffusion Parameter values in excess of the 0.1-15 sec⁻¹ range desired for the present process. Nevertheless, the required diffusivity can be achieved by severely steaming the zeolite so as to effect a controlled reduction in the micropore volume of the catalyst to not less than 50%, and preferably 50-90%, of that of the unsteamed catalyst. Reduction in micropore volume is monitored by measuring the n-hexane adsorption capacity of the zeolite, before and after steaming, at 90° C. and 75 torr n-hexane pressure.

Steaming to achieve the desired reduction in the micropore volume of the porous crystalline material can be effected by heating the material in the presence of steam at a temperature of at least about 950° C., preferably about 950 to about 1075° C., and most preferably about 1000 to about 1050° C. for about 10 minutes to about 10 hours, preferably from 30 minutes to 5 hours.

To effect the desired controlled reduction in diffusivity and micropore volume, it may be desirable to combine the porous crystalline material, prior to steaming, with at least one oxide modifier, preferably selected from oxides of the elements of Groups IIA, IIIA, IIIB, IVA, VA, VB and VIA of the Periodic Table (IUPAC version). Conveniently, said at least one oxide modifier is selected from oxides of boron, magnesium, calcium, lanthanum and preferably phosphorus. In some cases, it may be desirable to combine the porous crystalline material with more than one oxide modifier, for example a combination of phosphorus with calcium and/or magnesium, since in this way it may be possible to reduce the steaming severity needed to achieve a target diffusivity value. The total amount of oxide modifier present in the catalyst, as measured on an elemental basis, may be between about 0.05 and about 20 wt %, such as between about 0.1 and about 10 wt %, based on the weight of the final catalyst.

Where the modifier includes phosphorus, incorporation of a modifier in the alkylation catalyst is conveniently achieved by the methods described in U.S. Pat. Nos. 4,356,338; 5,110,776; 5,231,064; and 5,348,643, the entire disclosures of which are incorporated herein by reference. Treatment with phosphorus-containing compounds can readily be accomplished by contacting the porous crystalline material, either alone or in combination with a binder or matrix material, with a solution of an appropriate phosphorus compound, followed by drying and calcining to convert the phosphorus to its oxide form. Contact with the phosphorus-containing compound is generally conducted at a temperature of about 25° C. and about 125° C. for a time between about 15 minutes and about 20 hours. The concentration of the phosphorus in the contact mixture may be between about 0.01 and about 30 wt %.

Representative phosphorus-containing compounds, which may be used to incorporate a phosphorus oxide modifier into the catalyst include derivatives of groups represented by PX₃, RPX₂, R₂PX, R₃P, X₃PO, (XO)₃PO, (XO)₃P, R₃P═O, R₃P═S, RPO₂, RPS₂, RP(O)(OX)₂, RP(S)(SX)₂, R₂P(O)OX, R₂P(S)SX, RP(OX)₂, RP(SX)₂, ROP(OX)₂, RSP(SX)₂, (RS)₂PSP(SR)₂, and (RO)₂POP(OR)₂, where R is an alkyl or aryl, such as phenyl radical, and X is hydrogen, R, or halide. These compounds include primary, RPH₂, secondary, R₂PH, and tertiary, R₃P, phosphines such as butyl phosphine, the tertiary phosphine oxides, R₃PO, such as tributyl phosphine oxide, the tertiary phosphine sulfides, R₃PS, the primary, RP(O)(OX)₂, and secondary, R₂P(O)OX, phosphonic acids such as benzene phosphonic acid, the corresponding sulfur derivatives such as RP(S)(SX)₂ and R₂P(S)SX, the esters of the phosphonic acids such as dialkyl phosphonate, (RO)₂P(O)H, dialkyl alkyl phosphonates, (RO)₂P(O)R, and alkyl dialkylphosphinates, (RO)P(O)R₂, phosphinous acids, R₂POX, such as diethylphosphinous acid, primary, (RO)P(OX)₂, secondary, (RO)₂POX, and tertiary, (RO)₃P, phosphites, and esters thereof such as the monopropyl ester, alkyl dialkylphosphinites, (RO)PR₂, and dialkyl alkyphosphinite, (RO)₂PR, esters. Corresponding sulfur derivatives may also be employed including (RS)₂P(S)H, (RS)₂P(S)R, (RS)P(S)R₂, R₂PSX, (RS)P(SX)₂, (RS)₂PSX, (RS)₃P, (RS)PR₂, and (RS)₂PR. Examples of phosphite esters include trimethylphosphite, triethylphosphite, diisopropylphosphite, butylphosphite, and pyrophosphites such as tetraethylpyrophosphite. The alkyl groups in the mentioned compounds preferably contain one to four carbon atoms.

Other suitable phosphorus-containing compounds include ammonium hydrogen phosphate, the phosphorus halides such as phosphorus trichloride, bromide, and iodide, alkyl phosphorodichloridites, (RO)PCl₂, dialkylphosphoro-chloridites, (RO)₂PCl, dialkylphosphinochloroidites, R₂PCl, alkyl alkylphosphonochloridates, (RO)(R)P(O)Cl, dialkyl phosphinochloridates, R₂P(O)Cl, and RP(O)Cl₂. Applicable corresponding sulfur derivatives include (RS)PCl₂, (RS)₂PCl, (RS)(R)P(S)Cl, and R₂P(S)Cl.

Particular phosphorus-containing compounds include ammonium phosphate, ammonium dihydrogen phosphate, diammonium hydrogen phosphate, diphenyl phosphine chloride, trimethylphosphite, phosphorus trichloride, phosphoric acid, phenyl phosphine oxychloride, trimethylphosphate, diphenyl phosphinous acid, diphenyl phosphinic acid, diethylchlorothiophosphate, methyl acid phosphate, and other alcohol-P₂O₅ reaction products.

Representative boron-containing compounds, which may be used to incorporate a boron oxide modifier into the catalyst, include boric acid, trimethylborate, boron oxide, boron sulfide, boron hydride, butylboron dimethoxide, butylboric acid, dimethylboric anhydride, hexamethylborazine, phenyl boric acid, triethylborane, diborane, and triphenyl boron.

Representative magnesium-containing compounds include magnesium acetate, magnesium nitrate, magnesium benzoate, magnesium propionate, magnesium 2-ethylhexoate, magnesium carbonate, magnesium formate, magnesium oxylate, magnesium bromide, magnesium hydride, magnesium lactate, magnesium laurate, magnesium oleate, magnesium palmitate, magnesium salicylate, magnesium stearate, and magnesium sulfide.

Representative calcium-containing compounds include calcium acetate, calcium acetylacetonate, calcium carbonate, calcium chloride, calcium methoxide, calcium naphthenate, calcium nitrate, calcium phosphate, calcium stearate, and calcium sulfate.

Representative lanthanum-containing compounds include lanthanum acetate, lanthanum acetylacetonate, lanthanum carbonate, lanthanum chloride, lanthanum hydroxide, lanthanum nitrate, lanthanum phosphate, and lanthanum sulfate.

The porous crystalline material employed in the process of the disclosed embodiments may be combined with a variety of binder or matrix materials resistant to the temperatures and other conditions employed in the process. Such materials include active and inactive materials such as clays, silica and/or metal oxides such as alumina. The latter may be either naturally occurring or in the form of gelatinous precipitates or gels including mixtures of silica and metal oxides. Use of a material which is active, tends to change the conversion and/or selectivity of the catalyst and hence is generally not preferred. Inactive materials suitably serve as diluents to control the amount of conversion in a given process so that products can be obtained economically and orderly without employing other means for controlling the rate of reaction. These materials may be incorporated into naturally occurring clays, e.g., bentonite and kaolin, to improve the crush strength of the catalyst under commercial operating conditions. Said materials, i.e., clays, oxides, etc., function as binders for the catalyst. It is desirable to provide a catalyst having good crush strength because in commercial use it is desirable to prevent the catalyst from breaking down into powder-like materials. These clay and/or oxide binders have been employed normally only for the purpose of improving the crush strength of the catalyst.

Naturally occurring clays which can be composited with the porous crystalline material include the montmorillonite and kaolin family, which families include the subbentonites, and the kaolins commonly known as Dixie, McNamee, Georgia, and Florida clays or others in which the main mineral constituent is halloysite, kaolinite, dickite, nacrite, or anauxite. Such clays can be used in the raw state as originally mined or initially subjected to calcination, acid treatment, or chemical modification.

In addition to the foregoing materials, the porous crystalline material can be composited with a porous matrix material such as silica-alumina, silica-magnesia, silica-zirconia, silica-thoria, silica-beryllia, silica-titania, as well as ternary compositions such as silica-alumina-thoria, silica-alumina-zirconia, silica-alumina-magnesia, and silica-magnesia-zirconia.

The relative proportions of porous crystalline material and inorganic oxide matrix vary widely, with the content of the former ranging from about 1 to about 90% by weight and more usually, particularly when the composite is prepared in the form of beads, in the range of about 2 to about 80 wt % of the composite.

The conditions employed in the alkylation stage of the present process are not narrowly constrained but, in the case of the methylation of toluene, generally include the following ranges: (a) temperature between about 500 and about 700° C., such as between about 500 and about 600° C.; (b) pressure of between about 1 atmosphere and about 1000 psig (between about 100 and about 7000 kPa), such as between about 10 psig and about 200 psig (between about 170 and about 1480 kPa); (c) moles toluene/moles methanol (in the reactor charge) of at least about 0.2, such as from about 0.2 to about 20; and (d) a weight hourly space velocity (“WHSV”) for total hydrocarbon feed to the reactor(s) of about 0.2 to about 1000, such as about 0.5 to about 500 for the aromatic reactant, and about 0.01 to about 100 for the combined methanol reagent stage flows, based on total catalyst in the reactor(s).

The alkylation process can be conducted in any known reaction vessel with each of the methanol and aromatic feeds being injected into the reactor bed or beds in a single stage or in multiple stages. In one embodiment, the methanol feed is injected in stages into the reactor bed or beds at one or more locations downstream from the location of the injection of the aromatic reactant into the fluidized or fixed beds. For example, in a particular embodiment, a fluidized bed reactor may be used, and the aromatic feed can be injected into a lower portion of a single vertical fluidized bed of catalyst, with the methanol being injected into the bed at a plurality of vertically spaced intermediate portions of the bed and the product being removed from the top of the bed. Alternatively, the catalyst can be disposed in a plurality of vertically spaced catalyst beds, with the aromatic feed being injected into a lower portion of the first fluidized bed and part of the methanol being injected into an intermediate portion of the first bed and part of the methanol being injected into or between adjacent downstream catalyst beds.

Introducing methanol in stages improves the conversion of methanol with aromatics to produce higher order methyl aromatics, as taught in U.S. Pat. No. 6,642,426. The methanol feed can be distributed equally at each injection point or it may be distributed unequally, depending on the kinetics of the reaction and desired residence time. For example, a higher percentage of methanol can be injected into a lower injection point of a reactor bed if more residence time for the methanol is desired. Alternatively, a higher percentage of methanol can be injected at a higher injection point of the reactor if less residence time is required. Optimized methanol injection, which can be determined by one skilled in the art, improves the product yield and distribution and impacts certain conversion and selectivity targets, depending on reactor conditions, such as pressure, temperature, WHSV, and concentration of reactants and products, as well as the catalyst activity, selectivity and quantity.

Typically, water is co-injected with the methanol feed to reduce the methanol partial pressure and minimize side reactions of methanol to olefin by-products. Methanol injected without a diluent, such as water, generally leads to a higher amount of light gas by-products. Diluting the methanol decreases the amount available for the side reactions upon introduction into the reactor. In prior art processes, the methanol/water feed contains 25-30 wt % of water, preferably 27-29 of wt % water, based on the total weight of the methanol and water.

In one embodiment, unpurified methanol that contains at least 5 wt % water can be used as the alkylating agent. Using unpurified methanol that contains at least 5 wt % water is beneficial because it reduces the need to co-inject water with the methanol at least partially, if not completely. Thus, it should be appreciated that in at least some embodiments no additional water is co-injected along with the unpurified methanol. The unpurified methanol may contain about 5-35 wt % of water, preferably 20-35 wt % water. When the unpurified methanol contains enough water to dilute the methanol and reduce the methanol partial pressure and minimize side reactions of methanol to olefin by-products, no additional water is required to be co-injected with the unpurified methanol feed. While the unpurified methanol may contain trace oxygenates, such as ethanol, propanols, butanols, pentanols, dimethyl ether (DME), methyl formate, methyl acetate, acetone, and butanone, as one skilled in the art will know, such oxygenates will not adversely affect the alkylation reaction, as taught in U.S. Pat. No. 9,006,506, and therefore do not need to be removed from the unpurified methanol feed prior to injection into the reactor.

Using unpurified methanol has additional advantages when the aromatics plant is combined with a methanol production plant. Because a significant amount of water is tolerated, even preferred, for the methylation of toluene and/or benzene with methanol, the methanol may bypass the traditional methanol purification process (typically performed with multiple distillation or fractionation columns). Skipping this purification step allows for the elimination of at least one distillation tower, which means a savings of 3-10% of capital, or $10-20 million, in capital investment. Elimination of a tower also saves energy, which is estimated to be about 18.5 MW for 5 tons of methanol produced.

Crude methanol may also be used in other methanol conversion technologies, such as methanol-to-olefins (disclosed in U.S. Pat. Nos. 3,894,107; 3,928,483; 4,025,571; 4,423,274; and 4,433,189), methanol to gasoline (disclosed in U.S. Pat. Nos. 3,894,103; 3,894,104; 3,894,107; 4,035,430; and 4,058,576), methanol to aromatics, or any other methanol to hydrocarbon conversion process. The reactor for such conversion technologies may comprise fixed bed, moving bed or fluid bed, or other types of reactors suitable for the conversion of methanol.

Other methanol conversion technologies and catalysts may also deploy crude methanol such as those described in but not limited to: U.S. Pat. Nos. 8,623,321; 8,609,920; 8,609,919; U.S. Patent Publications Nos. 2005/0070749; 2006/0252633; 2011/0082025; 2011/0137099; 2011/0178356; 2011/0174692; 2011/0178354; 2012/0238789; 2012/0277509; 2013/0190546; 2013/0296622; 2013/0303820; 2014/0194663; 2014/0058157; 2015/0073187; PCT Publication Nos. 2003/059509; 2015/025327; 2015/184600; Chinese Patent Publication Nos. 102,964,201; 101,829,594; 101,885,662; 102,040,460; 102,040,459; 102,101,818; 102,205,251; 102,259,019; 102,259,018; 102,295,515; 102,335,622; 102,372,589; 102,372,588; 102,372,587; 102,372,586; 102,372,585; 102,372,584; 102,372,583; 102,372,582; 102,464,561; 102,464,560; 102,464,559; 102,464,558; 102,464,557; 102,464,550; 102,464,549; 102,464,540; 102,463,136; 102,463,085; 102,463,084; 102,463,072; 102,513,144; 102,600,887; 102,671,694; 102,688,771; 102,716,763; 102,731,243; 102,701,899; 102,746,099; 102,746,080; 102,744,111; 102,746,098; 102,746,095; 102,826,957; 102,872,904; 102,875,321; 102,875,320; 102,875,319; 102,875,317; 102,816,044; 102,909,064; 102,942,441; 102,951,993; 102,964,201; 103,113,182; 103,121,912; 103,121,911; 103,120,949; 103,263,946; 103,372,456; 103,418,421; 103,467,238; 103,588,611; 103,588,610; 103,588,601; 103,638,963; 103,664,492; 103,664,490; 103,664,488; 103,664,484; 103,708,496; 103,772,129; 103,769,246; 103,785,464; 103,785,463; 103,785,461; 103,801,402; 103,803,581; 103,804,112; 103,816,935; 103,878,014; 103,980,080; 104,096,589; 104,109,065; 104,117,385; 104,117,384; 104,128,198; 104,226,357; 104,051,639; 104,226,359; 104,230,633; 104,292,064; 104,275,209; 104,326,855; 104,342,198; 104,415,784; 104,437,599; 104,492,476; 104,447,158; 104,557,425; 104,557,376; 104,549,452; 104,710,268; 104,710,265; 104,874,418; 104,888,846; 104,945,219; 104,909,980; 105,080,593; 105,198,691; 105,214,714; 104,981,695; 105,272,798; 105,272,797; 105,315,120; 105,344,373; 105,439,790; 105,457,670; 105,115,333; and Japanese Patent Publication No. 2013/066884.

While the present invention has been described and illustrated by reference to particular embodiments, those of ordinary skill in the art will appreciate that the invention lends itself to variations and modifications not necessarily illustrated herein without departing from the spirit and scope of the invention. Accordingly, it is not intended that the scope of the claims appended hereto be limited to the examples and descriptions set forth herein but rather that the claims be construed as encompassing all the features of patentable novelty which reside herein, including all features which would be treated as equivalents thereof by those skilled in the art to which this disclosure pertains.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted. When numerical lower limits and numerical upper limits are listed herein, ranges from any lower limit to any upper limit are contemplated. The term “comprising” is synonymous with the term “including”. Likewise whenever a composition, an element or a group of components is preceded with the transitional phrase “comprising,” it is understood that we also contemplate the same composition or group of components with transitional phrases “consisting essentially of,” “consisting of,” “selected from the group of consisting of,” or “is” preceding the recitation of the composition, component, or components, and vice versa.

What we claim:

1. A process for the alkylation of toluene and/or benzene to produce paraxylene comprising contact of said toluene and/or benzene with an alkylating agent, in the presence of an alkylation catalyst in an alkylation reactor under alkylation conditions to produce an alkylation effluent comprising paraxylene, the improvement comprising using unpurified methanol as the alkylating agent, wherein the unpurified methanol contains at least 5 wt % of water, based on the weight of the unpurified methanol. to 2. The process of claim 1, wherein the unpurified methanol contains 20-35 wt % of water, based on the weight of the unpurified methanol.

3. The process of claim 2, wherein additional water is not co-injected with the unpurified methanol.

4. The process of claim 1, wherein the unpurified methanol contains less than 28 wt % of water, based on the weight of the unpurified methanol, and additional water is co-injected with the unpurified methanol to raise the amount of water to about 28 wt %, based on the total weight of the unpurified methanol and water.

5. The process of claim 4, wherein the unpurified methanol is injected in multiple stages axially along the reactor.

6. The process of claim 4, wherein the alkylation catalyst is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa).

7. The process of claim 6, wherein the alkylation catalyst is a medium-pore size aluminosilicate zeolite selected from the group consisting of ZSM-5, ZSM-11, ZSM-12, ZSM-22, ZSM-23, ZSM-35, and ZSM-48, optionally composited with an inorganic oxide matrix.

8. The process of claim 4, wherein the alkylation conditions comprise a temperature between about 500 and about 700° C., a pressure of between about 1 atmosphere and about 1000 psig (between about 100 and about 7000 kPa), a molar ratio of toluene/ methanol (in the reactor charge) of at least about 0.2 and a weight hourly space velocity (“WHSV”) for total hydrocarbon feed to the reactor(s) of about 0.2 to about 1000, based on total catalyst in the reactor(s).

9. The process of claim 4, wherein the alkylation effluent comprises at least 85 wt % of paraxylene.

10. A process for producing paraxylene, the process comprising:
a) contacting toluene and/or benzene with unpurified methanol in the presence of an alkylation catalyst in an alkylation reactor under alkylation conditions to produce an alkylation effluent comprising paraxylene, wherein the unpurified methanol contains at least 5 wt % of water, based on the weight of the unpurified methanol; and
b) recovering paraxylene from the alkylation effluent.

11. The process of claim 10, wherein the unpurified methanol contains 20-35 wt % of water, based on the weight of the unpurified methanol.

12. The process of claim 11, wherein additional water is not co-injected with the unpurified methanol.

13. The process of claim 10, wherein the unpurified methanol contains less than 28 wt % of water, based on the weight of the unpurified methanol, and additional water is co-injected with the unpurified methanol to raise the amount of water to about 28 wt %, based on the total weight of the unpurified methanol and water.

14. The process of claim 13, wherein the unpurified methanol is injected in multiple stages axially along the reactor.

15. The process of claim 13, wherein the alkylation catalyst is a porous crystalline material having a Diffusion Parameter for 2,2 dimethylbutane of about 0.1-15 sec⁻1 when measured at a temperature of 120° C. and a 2,2 dimethylbutane pressure of 60 torr (8 kPa).

16. The process of claim 15, wherein the alkylation catalyst is a medium-pore size aluminosilicate zeolite selected from the group consisting of ZSM-5, ZSM-11, ZSM-12, ZSM-22, ZSM-23, ZSM-35, and ZSM-48, optionally composited with an inorganic oxide matrix.

17. The process of claim 13, wherein the alkylation conditions comprise a temperature between about 500 and about 700° C., a pressure of between about 1 atmosphere and about 1000 psig (between about 100 and about 7000 kPa), a molar ratio of toluene/ methanol (in the reactor charge) of at least about 0.2 and a weight hourly space velocity (“WHSV”) for total hydrocarbon feed to the reactor(s) of about 0.2 to about 1000, based on total catalyst in the reactor(s).

18. The process of claim 13, wherein the alkylation effluent comprises at least 85 wt % of paraxylene.

19. The process of claim 13, wherein paraxylene is recovered from the alkylation effluent by simulated moving bed adsorption.
